(19) United States
(12) Patent Application Publication
(10) Pub. No.: US 2021/0127725 A1
(43) Pub. Date: May 6, 2021

(54) AN ISOMALTULOSE BASED SWEETENER

(71) Applicant: Evonik Operations GmbH, Essen (DE)

(72) Inventors: Thomas HAAS, Münster (DE); Thomas HÜLLER, Marl (DE); Michael RIMBACH, Greven (DE); Jan WOLTER, Düsseldorf (DE)

(21) Appl. No.: 16/635,820

(22) PCT Filed: Aug. 1, 2018
(86) PCT No.: PCT/EP2018/070865

§ 371 Date: Jan. 31, 2020

(30) Foreign Application Priority Data
Aug. 2, 2017 (EP) 17184484.8

(51) Int. Cl. A23L 27/30; A23L 29/30; A23G 3/42; C07D 309/10

(52) U.S. Cl. CPC A23L 27/34; A23L 29/37; A23V 2002/00; C07D 309/10; A23G 3/42

(57) ABSTRACT

The present invention relates to a sweetener comprising

20 wt. % to 75 wt. % of α-D-glucopyranosyl-1,6-D-sorbitol,

20 wt. % to 75 wt. % of α-D-glucopyranosyl-1,1-D-mannitol,

0.02 wt. % to 15 wt. % of α-D-glucopyranosyl-1,1-D-sorbitol,

0.02 wt. % to 15 wt. % of sorbitol

0.02 wt. % to 15 wt. % of mannitol,

less than 0.3 wt. % of sucrose, and

less than 0.0002 wt. % furan and derivatives thereof in each case relative to the total weight of dry matter of the sweetener and a method of producing the same.

### FIELD OF THE INVENTION

The present invention relates to an isomaltulose based sweetener, a method of producing such a sweetener and use of the sweetener in production of a confectionary. In particular, the sweetener may be considered a non-carcinogic sweetener in view of a negligible concentration of furan and/or derivatives thereof.

### BACKGROUND OF THE INVENTION

Isomalt (also called, isomaltitol, Palatinit®) is a sugar substitute, which is obtained from sucrose. It is usually produced in a two-stage process: first, sucrose is converted to isomaltulose (α-D-glucopyranosyl-1,6-fructose, also called Palatinose®) by rearrangement. The purified isomaltulose is then converted by catalytic hydrogenation to isomalt. In the hydrogenation of isomaltulose, two isomers form: α-D-glucopyranosyl-1,1-D-mannitol (designated 1,1-GPM hereinafter) and α-D-glucopyranosyl-1,6-D-sorbitol (designated 1,6-GPS hereinafter), of which isomalt essentially consists.

Something that all the known methods of isomerization of sucrose to isomaltulose have in common is that the sucrose is never completely converted—traces can always be detected, and for further processing of the isomaltulose to isomalt, separation of the sucrose that has not been isomerized must be carried out. The remaining sucrose is known to interfere with the hydrogenation process and therefore removed before hydrogenation is carried out.

WO/2011/076625 teaches a method where the isomaltulose mixture can be directly brought into contact with a catalyst for hydrogenation to be carried out without first removal of the remaining sucrose. This method not only speeds up the process of isomalt production, but also saves resources that may have been used during the sucrose separation.

In all these methods known in the art for isomalt production, furan is produced as a by-product in the final isomalt product. Furans are known, because of their strong taste properties (often a strong burned taste). However, furans since 1995, have also been classified as “possibly carcinogenic to humans” in Group 2B by the International Agency for Research on Cancer (IARC, 1995). In the recent years, furans have been found in a variety of heat treated food. Although, the actual of amount of furan exposure in human beings that may lead to toxicity and/or cancer is not known, it is generally accepted that the lesser of furans in the food and beverages human beings consume the better. In fact, it is recommended to keep the concentration of furans and/or derivatives thereof in the food and beverages human beings consume at negligible or trace concentrations.

Accordingly, there is a need in the art for a method of producing an isomaltulose based sweetener that is not only efficient but that also produces a sweetener that has negligible or trace concentrations of furans and/or derivatives thereof.

### DESCRIPTION OF THE INVENTION

The present invention attempts to solve the problems above by providing an isomaltulose based sweetener that has a trace concentration of furans and/or derivatives thereof and a means of making such a sweetener. In particular, the furan and/or derivative concentration may be less than 0.0002 wt. % relative to the total weight of dry matter of the sweetener. The sweetener may comprise other known components such as sorbitol, mannitol and sucrose found in most sweeteners.

According to one aspect of the present invention, there is provided a sweetener comprising

20 wt. % to 75 wt. % of α-D-glucopyranosyl-1,6-D-sorbitol,

20 wt. % to 75 wt. % of α-D-glucopyranosyl-1,1-D-mannitol,

0.02 wt. % to 15 wt. % of α-D-glucopyranosyl-1,1-D-sorbitol,

0.02 wt. % to 15 wt. % of sorbitol

0.02 wt. % to 15 wt. % of mannitol,

≤0.3 wt. % of sucrose, and

≤0.0002 wt. % furans and/or derivatives thereof.

In each case relative to the total weight of dry matter of the sweetener.

In particular, the sweetener comprises 0.00001-0.0002 wt. % furan and derivatives thereof.

According to another aspect of the present invention, there is provided a sweetener consisting of

20 wt. % to 75 wt. % of α-D-glucopyranosyl-1,6-D-sorbitol,

20 wt. % to 75 wt. % of α-D-glucopyranosyl-1,1-D-mannitol,

0.02 wt. % to 15 wt. % of α-D-glucopyranosyl-1,1-D-sorbitol,

0.02 wt. % to 15 wt. % of sorbitol

0.02 wt. % to 15 wt. % of mannitol,

less than 0.3 wt. % of sucrose, and

less than 0.0002 wt. % furan and derivatives thereof in each case relative to the total weight of dry matter of the sweetener.

In particular, the sweetener consists of 0.00001-0.0002 wt. % furan and derivatives thereof.

The sweetener according to any aspect of the present invention consists of the above mentioned substances in the stated wt. % in concentrations that may add up to 100.

The term “sweetener” used according to any aspect of the present invention refers to a mixture of compounds, which can be in liquid or solid form, crystalline or dissolved, optionally the sweetener may contain water and tastes sweet.

This sweetener has trace amounts of furans and/or derivatives thereof. This is advantageous as the sweetener according to any aspect of the present invention may be more appealing to the user as it has less carcinogenic components compared to the sweeteners known in the art.

For determination of furan and/or derivative concentration in an aqueous medium, any method known in the art may be used. In particular, quantitative 1H-NMR spectroscopy (qNMR) may be used. In one example, within the 1H-NMR spectrum a signal of methylene groups (multiplet at approx. 1.9 ppm) may be taken and quantified against quantification standard trimethylsilylpropanoic acid, with an assumption that one methylene group is present per furan molecule. In particular, the concentration of furans and/or derivatives thereof may be ≤0.0002 wt. % furans and/or derivatives thereof relative to the total weight of dry matter of the sweetener. More in particular, the concentration of furans and/or derivatives thereof may be about 0.00025, 0.00024, 0.00023, 0.00022, 0.00021, 0.0002, 0.00019, 0.00018, 0.00017, 0.00016, 0.00015, 0.00014, 0.00013, 0.00012, 0.00011, 0.00010, 0.00009, 0.00008, 0.00007, 0.00006, 0.00005, 0.00004, 0.00003, 0.00002, or 0.00001 wt. % relative to the total weight of dry matter of the sweetener. Even more in particular, the concentration of furans and/or derivatives thereof may be less than or equal to 0.0002, 0.00015, 0.0001, 0.00005 wt. % relative to the total weight of dry matter of the sweetener. The sweetener according to any aspect of the present invention must have at least trace or detectable amounts of furans and/or derivatives thereof. The concentration of furans and/or derivatives thereof in the sweetener according to any aspect of the present invention cannot have 0 wt. % relative to the total weight of dry matter of the sweetener.

The derivatives of furan according to any aspect of the present invention may be selected from the group consisting of 2,5-Bis-Hydroxymethyl Tetrahydrofuran, 2,5-Bis-Hydroxymethyl-3-Hydroxy-Tetrahydrofuran, Hexahydro-furo[3,2-b]furan-3-ol, furfural, furfuryl alcohol, tetrahydrofurfuryl alcohol, tetrahydroxyfuran, alkylfurans, halofurans, alkoxyfurans, furfuryl ester, furfuryl ethers, isosorbide and anhydromannitol. In particular, Hexahydro-furo[3,2-b]furan-3-ol may also be known as “Desoxy-Isosorbid” or (3R,3aR,6S,6aR)-Hexahydro-furo[3,2-b]furan-3,6-diol.

Another advantage of the sweetener according to any aspect of the present invention is that in comparison with the conventional isomalt (E953) and relative to 1,1-GPM, it is enriched with 1,6-GPS, which has a strong sweetening power and good dissolution in water.

For determination of the respective proportions by weight, it is possible to employ the methods described in the ISOMALT Specifications, elaborated within the scope of the 69th JECFA (2008), published in the FAO JECFA Monographs 5 (2008).

In one example, the sweetener according to any aspect of the present invention comprises less than 2.5 wt. %, in particular less than 0.3 wt. %, and more in particular, no detectable amounts of sucrose, relative to the total weight of dry matter of the sweetener.

According to a further aspect of the present invention, there is provided a confectionary comprising a sweetener according to any aspect of the present invention,

a carrier; and

flavouring.

The confectionary according to any aspect of the present invention may comprise a sweetener according to any aspect of the present invention that comprises trace amounts of furans and/or derivatives thereof allowing the confectionary not to compromise on the taste and yet be healthier than the products available on the market.

The term ‘confectionary’ used according to any aspect of the present invention is synonymous with sweets, candy, or sweet food. The confectionary may often comprise a sweetener. In particular, the confectionary according to any aspect of the present invention comprises the sweetener according to any aspect of the present invention. More in particular, the term may be divided into two broad and somewhat overlapping categories, bakers' confections and sugar confections. Confectioneries may include sweets, candy, candied nuts, chocolates, chewing gum, bubblegum, sweetmeats, pastillage, and other confections that are made primarily of sugar. Even more in particular, the confectionary may be selected from the group consisting of caramels, chocolates, divinity, dodol, dragé, fondant, fudge, halvah, hard candy (e.g. suckers, lollipops, jawbreakers (or gobstoppers), lemon drops, peppermint drops and disks, candy canes, rock candy, etc.), ice cream, jelly candies, liquorice, marshmallow, marzipan, mithai, persipan, tablet, taffy or chews, toffee and the like. The confectionary according to any aspect of the present invention may comprise at least these three ingredients: (a) sweetener according to any aspect of the present invention, (b) flavouring, and (c) carrier at varying concentrations. In particular, the concentration of (a) sweetener in the confectionary according to any aspect of the present invention may be about 99 wt. %. More in particular, the concentration of sweetener may be 10-99, 20-99, 30-99, 40-99, 50-99, 60-99, 70-99, 80-99, 90-99, 10-90, 20-90, 30-90, 40-90, 50-90, 60-90, 70-90, or 80-90 wt. % relative to the weight of the confectionary.

The confectionary according to any aspect of the present invention may further comprise a flavouring and a carrier. The flavouring may be selected from the group consisting of vanilla, hazelnut, cinnamon, bergamot, mint, cocoa, mocha, caramel, karob, citrus, berry, menthol, and mixtures thereof.

The amount of flavouring in the confectionary according to any aspect of the present invention may be varied in order to obtain the result desired in the final product. In general, the flavouring may be present in amounts of about 0.02% to about 5%, more in particular, from about 0.05% to about 3%, and even more in particular, from about 0.50% to about 1.8%, by weight of the confectionary.

The carrier used according to any aspect of the present invention may be selected from the group consisting of a monosaccharide, a disaccharide, inulin, fructooligosaccharide (FOS) and other fibers, maltodextrins, a sugar alcohol, a digestion resistant maltodextrin, a sugar polymer, and mixtures thereof. The amount of carrier employed may be determined by the amount of the flavouring and sweetener found in the confectionary according to any aspect of the present invention. In particular, the amount of carrier used according to any aspect of the present invention in combination with the wt. % of sweetener and flavouring add up to 100. Accordingly, the concentration of carrier may be 0.05% to about 99% by weight of the confectionary.

According to yet another aspect of the present invention, there provided a method of producing at least one sweetener comprising less than 0.0002 wt. % furan and derivatives thereof, wherein the method comprises

(a) hydrogenating a carbohydrate mixture in the presence of a ruthenium (Ru) catalyst immobilised on an active charcoal support,

wherein the carbohydrate mixture comprises isomaltulose and sucrose and the pH of the carbohydrate mixture is maintained at ≥3.

According to one aspect of the present invention, there provided a method of producing at least one sweetener comprising less than 0.0002 wt. % furan and/or derivatives thereof, wherein the method comprises

(a) hydrogenating a carbohydrate mixture in the presence of a ruthenium (Ru) catalyst immobilised on an aluminium support,

wherein the carbohydrate mixture comprises isomaltulose and sucrose and the pH of the carbohydrate mixture is maintained at ≥3.

In this aspect the aluminium support is an Al₂O₃ support. With the use of a ruthenium (Ru) catalyst immobilised on an active charcoal support or aluminium support, the amount of furan and/or derivatives thereof formed is reduced and less than that formed when Ru catalysts immoblised on other known supports.

The method according to any aspect of the present invention has the advantage of making the sweetener directly available as a product. No further steps may be needed after carrying out the method according to any aspect of the present invention before using the sweetener.

A further advantage of the method according to any aspect of the present invention is that it can be carried out at relatively low temperatures and pressures and therefore saves energy and resources.

The carbohydrate mixture comprising isomaltulose and sucrose may also comprise trehalulose, fructose and glucose and/or other polysaccharides with hydrogen, characterized in that the reaction is carried out in the presence of at least one catalyst, which is based on ruthenium (Ru) immobilised on an active charcoal support. In one example, the carbohydrate mixture can contain water and the method according to any aspect of the present invention is then carried out in an aqueous medium. In particular, the carbohydrate mixture therefore contains 20 wt. % to 80 wt. %, more in particular 30 wt. % to 70 wt. %, even more in particular, 40 wt. % to 60 wt. % of water relative to the total carbohydrate mixture.

The carbohydrate mixture used in the method according to any aspect of the present invention may be obtainable by the enzymatic reaction of sucrose-containing aqueous solutions, for example aqueous solutions of sugar from sugar beet or sugar cane, with isomaltulose synthases.

Suitable isomaltulose synthases may be selected from the group consisting of Enterobacter sp. strain FMB1, Erwinia rhapontici, Klebsiella planticola strain UQ14S, Klebsiella pneumoniae NK33-98-8, Klebsiella sp. LX3, Pantoea dispersa UQ68J, Protaminobacter ruber Z12, Protaminobacter rubrum, Pseudomonas mesoacidophila MX-45, Serratia plymuthica. In particular, carbohydrate mixtures obtainable by the enzymatic reaction of sucrose-containing, aqueous solutions with isomaltulose synthases from Protaminobacter rubrum, in particular of the strain Protaminobacter rubrum CBS 574.77, can be used advantageously in the method according to any aspect of the present invention.

The sucrose contained in the carbohydrate mixture may therefore be considered residual sucrose. In particular, the carbohydrate mixture used in the method according to any aspect of the present invention may contain 0.01 wt. % to 15 wt. %, 0.1 wt. % to 5 wt. % or 0.2 wt. % to 2 wt. % of sucrose relative to the dry weight of the total carbohydrate mixture.

The carbohydrate mixture used in the method according to any aspect of the present invention may comprise at least 70 wt. %, 80 wt. %, 90 wt. % of isomaltulose relative to the dry weight of the total carbohydrate mixture.

The carbohydrate mixture used in the method according to any aspect of the present invention may further comprise 0.02 wt. % to 30 wt. %, 0.1 wt. % to 20 wt. %, or 0.2 wt. % to 10 wt. % of trehalulose relative to the dry weight of the total carbohydrate mixture.

In the method according to any aspect of the present invention, both the isomaltulose and trehalulose when present may be hydrogenated catalytically by hydrogen to 1,1-GPM and 1,6-GPS and optionally to 1,1-GPS. The sucrose may be cleaved to fructose and glucose and these are then hydrogenated to mannitol and sorbitol.

Therefore, the reaction in the method according to any aspect of the present invention corresponds to a catalytic hydrogenation accompanied by cleavage of the sucrose to fructose and glucose. It is therefore useful for the cleavage of the sucrose and the hydrogenation of the other carbohydrates present to take place simultaneously.

In particular, the pH may be maintain in the range of 3 to 5, inclusive. In particular, the pH may be selected from the group consisting of about 3, 3.5, 4, 4.5 and 5. In one example, the pH may be about 3. In another example, the pH may be about 3.5 and in a further example, the pH may be about 4. A pH less than 3 may further lead to increase in furans and/or derivatives formed. The pH of the carbohydrate mixture may be controlled by adding a suitable acid.

In one example, the carbohydrate mixture in the method according to any aspect of the present invention may be heated to a temperature in the range of 90 to 150° C. before step (a) is carried out. In particular, the time taken for increasing the temperature of the carbohydrate mixture may be about ≤60 minutes. In particular, the temperature of the carbohydrate mixture may be increased to 90-120° C. in about 60, 55, 50, 45, 40, 35, 30, 29, 28, 27, 26, 25, 24, 23, 22, 21, 20, 19, 18, 17, 16, 15, 14, 13, 12, 11 or 10 minutes. A longer period used to increase the carbohydrate temperature to at least 90° C. may result in an increase in the amount of furans and/or derivatives thereof. It may therefore be important to maintain the time required to increase the temperature of the carbohydrate mixture used in the method according to any aspect of the present invention to 90-120° C. in a period less than or equal to 60 minutes. In particular, the temperature of the carbohydrate mixture may be increased to at least 90-120° C. in less than or equal to 30 minutes. In another example, the temperature of the carbohydrate mixture may be increased to at least about 95, 100, 105, 110, 115 or 120° C. The heating may be carried out in the presence of hydrogen gas for less furan and/or derivative production.

In a further example, the pH of the carbohydrate mixture according to any aspect of the present invention may be about 3.5 and the heating of the carbohydrate mixture to at least 90° C. may be carried out in less than ≤30 minutes. The temperature of the carbohydrate mixture then be maintained at 90-120° C. for the rest of the hydrogenation reaction

The pressure during the hydrogenation reaction may be at least 15 bar. In particular, the pressure may be at least 30 bar or 40 bar. Values between 40 bar and 150 bar, in particular between 40 bar and 90 bar, for example in the range from about 50 to 60 bar, are especially preferable. Even more in particular the pressure may be about 50 bar.

As used herein, the terms “about”, as applied to the concentrations of different components of the sweetener, temperatures and pH refer to a range of values that are similar to the stated reference value for that condition. In certain examples, the term “about” refers to a range of values that fall within 25, 20, 19, 18, 17, 16, 15, 14, 13, 12, 11, 10, 9, 8, 7, 6, 5, 4, 3, 2, 1 percent or less of the stated reference value for that condition. For example, a temperature employed during the method according to any aspect of the present invention when modified by “about” includes the variation and degree of care typically employed in measuring in an experimental condition in production plant or lab. For example, the temperature when modified by “about” includes the variation between batches in multiple experiments in the plant or lab and the variation inherent in the analytical method. Whether or not modified by “about,” the amounts include equivalents to those amounts. Any value stated herein and modified by “about” can also be employed in the present invention as the amount not modified by “about.”

The method according to any aspect of the present invention may be carried out until sucrose can no longer be detected in the sweetener obtained according to any aspect of the present invention.

The sweetener obtained by the method according to any aspect of the present invention, may be in liquid form, and may be converted to the dry form using any method known in the art. In particular, the water present as solvent can be removed using an evaporator or a dryer, for example a down-flow evaporator or a drum dryer or a spray dryer.

It may be advantageous for the sweetener obtained to be further processed with additional purification or enrichment and/or depletion steps.

Thus, it may be advantageous to lower the content of mannitol by applying another crystallization step, for example to 0.02-15 wt. %, particularly to 0.1-10 wt. %, or 0.2-2.9 wt. % relative to the dry weight of the sweetener; this is easily possible owing to the low water solubility of mannitol.

It may also be advantageous to include a washing step before the catalyst is used. Washing may be carried out using any washing medium known in the art. In particular, washing may be carried our using water, desalted water and combinations thereof. The washing of the catalyst before use in the method according to any aspect of the present invention may lower the chloride content to ≤5 ppm of the catalyst. This step also reduces the overall furan and/or derivative production in the method according to any aspect of the present invention. In one example, the washing step may result in a chloride free catalyst.

The method according to any aspect of the present invention may further comprise a step of processing the final product (i.e. the sweetener according to any aspect of the present invention) to remove any furan and/or derivative found in the final product. Any method known in the art may be used for processing of the final product. Some known methods that can be used to process the final products may include the use of activated carbon treatment to remove furans, use of hydrophobic extractants such as hydrocarbons (for example hexadecane) that may be brought into contact with the final product for extraction of furans and/or derivatives thereof and the like.

According to another aspect of the present invention, there may be provided use of the sweetener according to any aspect of the present invention for producing a confectionary.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

Methods and Materials

Unless stated differently, respective ruthenium catalysts as listed below were washed to make them chloride free using desalted water until chloride content was below 5 ppm (measured via ion exchange chromatography) before carrying out hydrogenation experiments.

345.0 g solution containing 145.5 g isomaltulose (-hydrate), 10.4 g sucrose and 189.1 g water (corresponding to 40 w. % isomaltulose and 3 w. % sucrose in aqueous solution or 93 w. % isomaltulose dry matter and 7 w. % sucrose dry matter) was hydrogenated at 90° C. and 50 bar hydrogen using 25 g of 2 w. % ruthenium catalyst with respective carrier. The ruthenium content was kept constant at 0.5 g. The catalyst volume varied according bulk density of respective carrier material.

Educt solution as well as sample taken after 2 h reaction was analyzed using HPLC to determine turnover and selectivity. The content of furans was determined using quantitative 1H-NMR spectroscopy (qNMR). Within the 1H-NMR spectrum a signal of methylene groups (multiplet at approx. 1.9 ppm) was taken and quantified against quantification standard trimethylsilylpropanoic acid, assuming one methylene group per furan molecule.

### Example 1

Ruthenium catalyzed hydrogenation of isomaltulose solution containing sucrose on activated carbon carrier

| | | | | Selectivity | Furan | pH product |
|---|---|---|---|---|---|---|
| | Sucrose | Turnover | Turnover | 1,6-GPS + | content | solution |
| | 0 h/2 h | sucrose | isomaltulose | 1,1-GPM | after 2 h | after 2 h |
| Catalyst | [% TS] | [%] | [%] | [%] | [mg/kg] | [—] |
| Evonik 2% | 7.0/0.8 | 88.8 | 100 | 99.1 | 0.5 | 3.6 |
| Ru/C | | | | | | |
| Noblyst 3000 | | | | | | |

### Example 2

Ruthenium catalyzed hydrogenation of isomaltulose solution containing sucrose on Al2O3 carrier

| | | | | Selectivity | Furan | pH product |
|---|---|---|---|---|---|---|
| | Sucrose | Turnover | Turnover | 1,6-GPS + | content | solution |
| | 0 h/2 h | sucrose | isomaltulose | 1,1-GPM | after 2 h | after 2 h |
| Catalyst | [% TS] | [%] | [%] | [%] | [mg/kg] | [—] |
| Evonik 2% | 7.0/1.0 | 87.0 | 99.2 | 98.8 | 23 | 3.6 |
| RU/Al2O3 | | | | | | |
| Noblyst 3001 | | | | | | |

### Example 3

Ruthenium catalyzed hydrogenation of isomaltulose solution containing sucrose on TiO2 carrier

| | | | | Selectivity | Furan | pH product |
|---|---|---|---|---|---|---|
| | Sucrose | Turnover | Turnover | 1,6-GPS + | content | solution |
| | 0 h/2 h | sucrose | isomaltulose | 1,1-GPM | after 2 h | after 2 h |
| Catalyst | [% TS] | [%] | [%] | [%] | [mg/kg] | [—] |
| Evonik 2% | 7.0/3.5 | 53.3 | 98.1 | 99.8 | 39 | 3.5 |
| Ru/TiO2 | | | | | | |
| Aerolyst 7711 | | | | | | |

### Example 4

Ruthenium catalyzed hydrogenation of isomaltulose solution containing sucrose on activated carbon carrier at pH<3

40 w. % lsomaltulose 3 w. % sucrose solution was hydrogenated at 90° C. and 50 bar hydrogen on activated carbon carrier, which was not washed chloride free before use.

| | | | | Selectivity | Furan | pH product |
|---|---|---|---|---|---|---|
| | Sucrose | Turnover | Turnover | 1,6-GPS + | content | solution |
| | 0 h/2 h | sucrose | isomaltulose | 1,1-GPM | after 2 h | after 2 h |
| Catalyst | [% TS] | [%] | [%] | [%] | [mg/kg] | [—] |
| Evonik 2% | 7.0/0.0 | 100 | 98.5 | 96.2 | 13 | 2.6 |
| Ru/C | | | | | | |
| Noblyst 3000 | | | | | | |

The content of furans increased significantly by factor 25 compared to example 1, accompanying acidic cleavage of sucrose yet less selective and incomplete turnover of isomaltulose to GPM/GPS.

### Example 5

Ruthenium catalyzed hydrogenation of isomaltulose solution containing sucrose with heating up in ≤60 minutes

40 w. % Isomaltulose 3 w. % sucrose solution was hydrogenated at 90° C. and 50 bar hydrogen on TiO2 carrier in varying quantity and heating up time, respectively.

| | | | | | | Selectivity | Furan | pH product |
|---|---|---|---|---|---|---|---|---|
| | Catalyst | Heating | Sucrose | Turnover | Turnover | 1,6-GPS + | content | solution |
| | amount | up time | 0 h/2 h | sucrose | isomaltulose | 1,1-GPM | after 2 h | after 2 h |
| Catalyst | [g] | [min] | [% TS] | [%] | [%] | [%] | [mg/kg] | [—] |
| Evonik 2% | 35 | 60 | 7.0/2.8 | 62.0 | 100 | 99.3 | 49 | 3.5 |
| Ru/TiO2 | 53 | 20 | 7.0/1.6 | 78.2 | 100 | 98.7 | 32 | 3.8 |
| Aerolyst 7711 | 89 | 20 | 7.0/0.0 | 97.4 | 100 | 98.2 | 73 | 3.8 |

Even though less ruthenium catalyst was used, the formation of furans increased with extended heating up time of the reactor.

1-15. (canceled)

16. A sweetener comprising:
20 wt. % to 75 wt. % of α-D-glucopyranosyl-1,6-D-sorbitol,
20 wt. % to 75 wt. % of α-D-glucopyranosyl-1,1-D-mannitol,
0.02 wt. % to 15 wt. % of α-D-glucopyranosyl-1,1-D-sorbitol,
0.02 wt. % to 15 wt. % of sorbitol,
0.02 wt. % to 15 wt. % of mannitol,
less than 0.3 wt. % of sucrose, and
0.00001-0.0002 wt. % furan and derivatives thereof,
in each case relative to the total weight of dry matter of the sweetener.

17. The sweetener of claim 16, wherein the concentration of furan and derivatives thereof is less than 0.0001 wt. % relative to the total weight of dry matter of the sweetener.

18. The sweetener of claim 16, wherein the derivatives of furan are selected from the group consisting of 2,5-Bis-Hydroxymethyl Tetrahydrofuran, 2,5-Bis-Hydroxymethyl-3-Hydroxy-Tetrahydrofuran, Hexahydro-furo[3,2-b]furan-3-ol, furfural, furfuryl alcohol, tetrahydrofurfuryl alcohol, tetrahydroxyfuran, alkylfurans, halofurans, alkoxyfurans, furfuryl ester, furfuryl ethers, isosorbide and anhydromannitol.

19. The sweetener of claim 16, wherein said sweetener consists of:
0 wt. % to 75 wt. % of α-D-glucopyranosyl-1,6-D-sorbitol,
20 wt. % to 75 wt. % of α-D-glucopyranosyl-1,1-D-mannitol,
0.02 wt. % to 15 wt. % of α-D-glucopyranosyl-1,1-D-sorbitol,
0.02 wt. % to 15 wt. % of sorbitol,
0.02 wt. % to 15 wt. % of mannitol,
less than 0.3 wt. % of sucrose, and
0.00001-0.0002 wt. % furan and derivatives thereof, in each case relative to the total weight of dry matter of the sweetener.

20. A confectionary comprising the sweetener of claim 16,
a carrier; and
flavouring.

21. The confectionary of claim 20, wherein the concentration of furan and derivatives in the sweetener, is less than 0.0001 wt. % relative to the total weight of dry matter of the sweetener.

22. The confectionary of claim 20, wherein the derivatives of furan in the sweetener are selected from the group consisting of 2,5-Bis-Hydroxymethyl Tetrahydrofuran, 2,5-Bis-Hydroxymethyl-3-Hydroxy-Tetrahydrofuran, Hexahydro-furo[3,2-b]furan-3-ol, furfural, furfuryl alcohol, tetrahydrofurfuryl alcohol, tetrahydroxyfuran, alkylfurans, halofurans, alkoxyfurans, furfuryl ester, furfuryl ethers, isosorbide and anhydromannitol.

23. The confectionary of claim 20, wherein the flavouring is selected from the group consisting of vanilla, hazelnut, cinnamon, bergamot, mint, cocoa, mocha, caramel, karob, citrus, berry, menthol, and mixtures thereof.

24. The confectionary of claim 20, wherein the carrier is selected from the group consisting of a monosaccharide, a disaccharide, inulin, fructooligosaccharide (FOS) and other fibers, maltodextrins, a sugar alcohol, a digestion resistant maltodextrin, a sugar polymer, and mixtures thereof.

25. The confectionary of claim 24, wherein the flavouring is selected from the group consisting of vanilla, hazelnut, cinnamon, bergamot, mint, cocoa, mocha, caramel, karob, citrus, berry, menthol, and mixtures thereof.

26. A method of producing at least one sweetener comprising 0.00001-0.0002 wt. % furan and derivatives thereof, wherein the method comprises a) hydrogenating a carbohydrate mixture in the presence of a ruthenium (Ru) catalyst immobilised on an active charcoal support, wherein the carbohydrate mixture comprises isomaltulose and sucrose and the pH of the carbohydrate mixture is maintained in the range of 3 to 5.

27. The method of claim 26, wherein the pH is about 3.

28. The method of claim 26, wherein the pH is about 4.

29. The method of claim 26, wherein the carbohydrate mixture is heated to a temperature in the range of 90 to 120° C. before step (a) is carried out and the heating of the carbohydrate mixture is carried out in ≤60 minutes.

30. The method of claim 29, wherein the heating of the carbohydrate mixture is carried out in ≤30 minutes.

31. The method of claim 26, wherein the pH of the carbohydrate mixture is about 4 and the heating of the carbohydrate mixture to about 90° C. is carried out in ≤30 minutes.

32. The method of claims 26, wherein hydrogenation is accompanied by cleavage of sucrose.

33. The method of claim 32, wherein the pH of the carbohydrate mixture is about 4 and the heating of the carbohydrate mixture to about 90° C. is carried out in ≤30minutes.
